(19) United States
(12) Patent Application Publication
(10) Pub. No.: US 2005/0171483 A1
(43) Pub. Date: Aug. 4, 2005

(54) Needle cover extractor

(76) Inventor: Dennis Williams, Winchester, VA (US)

Correspondence Address: Richard C. Litman;LITMAN LAW OFFICES, LTD., P.O. Box 15035, Arlington, VA, 22215, US

(21) Appl. No.: 10/769,876

(22) Filed: Feb. 3, 2004

(51) Int. Cl. A61M 5/00

(52) U.S. Cl. 604/187

(57) ABSTRACT

The needle cover extractor is a semi-rigid clip having a notched slot designed to abut and closely surround a needle hub of a syringe just below a needle cover. The notched slot defines two legs and a connecting body. The notched slot can be made with varying dimensions to fit the particular needle hub being used. The extractor has a top surface and a bottom surface that is interchangeable and indistinguishable from one another and thus permits a user to utilize the extractor without regard to the extractor's position on the needle hub. In use, the user can simultaneously apply upward and downward force on the extractor or individually apply upward or downward force on the extractor to dislodge the needle cover from the needle hub. As an alternative arrangement for the slot, the slot may be made to have a substantially U-shaped configuration.

### BACKGROUND OF THE INVENTION

1. Field of the Invention

The present invention relates to accessories for medical devices, and more specifically, to an accessory for dislodging a needle cap mounted on the needle hub of a syringe.

2. Description of the Related Art

Individuals who administer drugs and work with hypodermic needles not only recognize the importance of maintaining hygienic conditions, but also realize the possibility of accidentally pricking oneself with the needle. Maintaining sanitary conditions during the administration of drugs, especially when using hypodermic needles, is essential to prevent infection and transference of disease. Today most hypodermic needles are manufactured with needle covers to reduce the possibility of contaminating the needle and pricking oneself. In order to administer drugs through hypodermic needles, the needle cover must be removed. Generally, removing needle covers is not a difficult task, however, at times the grip of the needle cover to a needle hub on a syringe is so strong that a great deal of effort is required from the drug administrator to uncover the needle. Thus a device able to help remove the needle cover from the syringe without contaminating or damaging the needle and without pricking oneself is desired.

Japanese Patent Number 6-121836, published on May 6, 1994, shows a lever mounted on one edge of a syringe cover by a hinge. The lever has a handle portion for a user's fingers to permit capping and uncapping of the cover on the syringe. To uncap the syringe, the lever is pushed down and wedged against the side of the syringe until the needle cover can be easily removed. An alternative, embodiment shows a wedge shaped device having a handle. The wedged portion is slid below the needle cap and the thumb rests on the handle to push the device up and thus dislodge the needle cap.

A number of devices have been developed that wedge the needle cover to remove it from over the syringe. U.S. Pat. No. 4,596,562, issued to Vernon on Jun. 24, 1986, describes a two-ended plate-like member for handling syringe needle covers. At one end, the device has a “v” shaped channel to help remove and replace cylindrical syringe needle covers. The “v” shaped channel works by a user wedging the cylindrical syringe needle down into the “v” so the handle grips the needle cover. At the opposite end there are a number of holes of varying diameters to help the user to uncap and cap a tapered needle cover.

U.S. Pat. No. 4,717,386, issued to Simmons on Jan. 5, 1988, describes a safety device for uncapping and recapping a protective sheath on a needle. The device is a paddle shaped member including a hand shield section. The device has a detent for receiving the sheath on a needle.

U.S. Pat. No. 5,156,426, issued to Graves on Apr. 18, 1991, describes a needle guard. The device is a hand held tool having holes to receive and remove a needle cover. The device is preferably seven to ten inches in length, about one inch wide and about one eight to one sixteenth of an inch thick. A number of finger grooves are disposed on a handle of the device for easy gripping.

U.S. Pat. No. 4,915,698, issued to Levenson on Apr. 10, 1990, describes a device for removing and replacing a needle cover on a syringe. The device has a base and an attached tubular member extending from the base. The tubular member comprises a cylindrical cavity having an open end with a partial lip jutting into the open end. In use, the capped syringe is inserted into the cylindrical cavity through the open end of the device and the cap is removed from the syringe by engaging the cap against the partial lip. To recap the syringe, the needle is inserted back into the cavity, which holds the cap until the cap sets on the needle.

A number of needle covers have been developed that pivot about a needle body to uncap and recap the needle. U.S. patent application Ser. No. 2003/0181860, published to Swenson on Sep. 25, 2003, describes a biased needle shield assembly, having a needle shield and a biasing member. The needle shield is pivotally connected to a needle holder having a lever disposed at the end adjacent the needle holder. The biasing member is disposed on the needle holder adjacent the lever, between the needle holder and the needle shield. In use, the lever is pivoted about a spring against the biasing member to lift the needle cover and expose the needle. After the needle has been used, the lever is pivoted back to its original position to conceal the needle.

U.S. Pat. No. 4,886,503, issued to Miller on Dec. 12, 1989, describes a covered needle for a disposable syringe. The cover is pivotably connected to the syringe to cover the needle. The cover has a long narrow opening for the needle to pass through when the cover is pivoted to one side to expose the needle.

U.S. Pat. No. 5,055,102, issued to Sitnik on Oct. 8, 1991, describes a swing-away disposable syringe needle cover. The cover is a tapered tube that covers both the needle and the hub. To expose the needle, the cover is pivoted to one side and the needle exits out a longitudinal slit disposed on the cover.

U.S. Pat. No. 6,077,253, issued to Cosme on Jun. 20, 2000, describes a safety needle assembly having a retractable needle shield for covering and uncovering the tip of a needle. The retractable needle shield works by sliding a retractable hollow tube about a stationary hollow tube to permit the retractable hollow tube to slip in and out of the stationary tube.

A number of needle covers have been disclosed in: U.S. Pat. No. 4,636,201, issued to Ambrose et al. on Jan. 13, 1987 (a plastic sheath cover in conjunction with a rubber needle cover is placed over the needle to provide a protective barrier for the needle); U.S. Pat. No. 5,591,134, issued to Shu on Jan. 7, 1997 (a cylindrical-shaped protective needle cover having two pivotally connected halves where one half is rotated open to position a needle within the cover); and U.S. Pat. No. 5,084,028, issued to Kennedy et al. on Jan. 28, 1992 (a needle cover and dispenser).

Other needle covers and safety devices have been disclosed in British Patent Number 2,198,644, published on Jun. 22, 1988 (holder for hypodermic needle sheath); Japanese Patent Number 2003-79726, published on Mar. 18, 2003 (needle cover for a syringe for that utilizes anti-skid projections); U.S. patent application Ser. No. 2003/0120218, published to Asbaghi on Jun. 26, 2003 (safety device for a sheathed, pre-filled injection syringe); U.S. Pat. No. 6,254,575, issued to Thorne, Jr. et al., on Jul. 3, 2001 (reaccessible medical needle safety devices and methods); and U.S. Pat. No. 4,850,996, issued to Cree on Jul. 25, 1989 (a chambered safety needle sheath that surrounds the needle and steadies the needle as it is pressed into flesh).

None of the above inventions and patents, taken either singly or in combination, is seen to describe the instant invention as claimed. Thus a needle cover extractor solving the aforementioned problems is desired.

### SUMMARY OF THE INVENTION

The needle cover extractor is a semi-rigid clip having a notched slot designed to abut and closely surround a needle hub of a syringe just below a needle cover. The notched slot defines two legs and a connecting body. The notched slot can be made with varying dimensions to fit the particular needle hub being used. The extractor has a top surface and a bottom surface that is interchangeable and indistinguishable from one another and thus permits a user to utilize the extractor without regard to the extractor's position on the needle hub. In use, the user can simultaneously apply upward and downward force on the extractor or individually apply upward or downward force on the extractor to dislodge the needle cover from the needle hub. As an alternative arrangement for the slot, the slot may be made to have a substantially U-shaped configuration.

### BRIEF DESCRIPTION OF THE DRAWINGS

FIG. 1A is an exploded, perspective view of a needle cover extractor according to the present invention and a syringe.

FIG. 1B is an environmental, perspective view of a needle cover extractor according to the present invention disposed on a syringe.

FIG. 2A is a perspective view of a needle cover extractor according to the present invention.

FIG. 2B is a plan view of a needle cover extractor according to the present invention.

FIG. 2C is a plan view a needle cover extractor according to the present invention having a substantially U-shaped slot.

FIG. 3A is a environmental perspective view of a needle cover extractor according to the present invention being forced in an upward direction to dislodge a needle cover up off of a needle hub.

FIG. 3B is a environmental perspective view of a needle cover extractor according to the present invention being forced in a downward direction to dislodge a needle cover up off of a needle hub.

FIG. 4A is a perspective view of an alternative embodiment of the needle cover extractor of the present invention.

FIG. 4B is a plan view of an alternative embodiment of the needle cover extractor of the present invention.

FIG. 4C is a plan view of an alternative embodiment of the needle cover extractor of the present invention having a substantially U-shaped slot.

FIG. 5 is an environmental, perspective view of an alternative embodiment of the needle cover extractor of the present invention being forced in a simultaneous upward and downward direction to dislodge a needle cover up off of a needle hub.

Similar reference characters denote corresponding features consistently throughout the attached drawings.

### DETAILED DESCRIPTION OF THE PREFERRED EMBODIMENTS

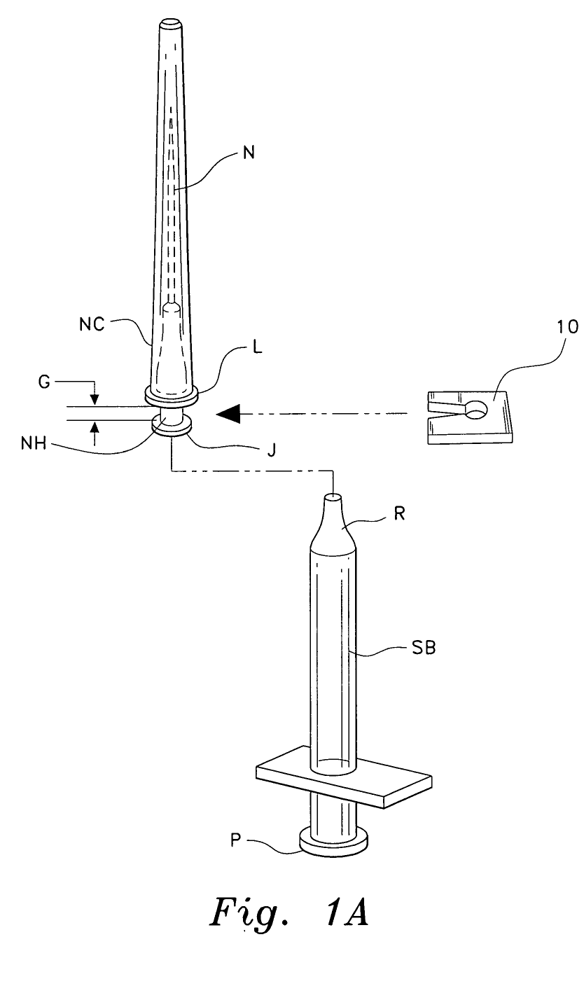

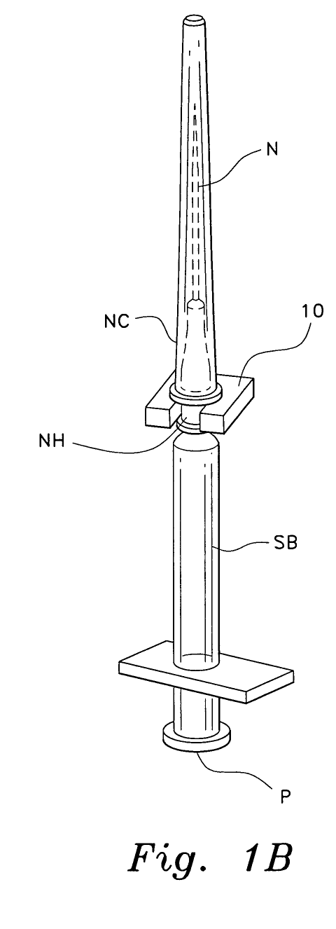

The present invention is a needle cover extractor, designated as 10 in the drawings. The extractor 10 is a clip designed to fit around a needle hub NH of a syringe, see FIGS. 1A and 1B. The syringe comprises a plunger P, a syringe body SB, a protuberance R, the needle hub NH, a needle N and a needle cover NC. The syringe shown in the figures is a two-part needle-bearing syringe that must be assembled before use. One part of the syringe is the needle N, which is mounted on the needle hub NH and is encased by the needle cover NC. The second part of the syringe is the plunger P, the syringe body SB and the protuberance R.

To assemble the syringe, the needle hub NH must be pressed or threaded onto the protuberance R of the syringe. Whether the needle hub NH and protuberance R is threaded or smooth determines if the needle hub NH will be pressed or threaded on the protuberance R. The type of needle hub NH and protuberance R also determines how easily the needle hub NH will come off of the protuberance R when one is only trying to remove the needle cover NC from the needle hub NH. To facilitate the removal of the needle cover NC the extractor 10 is disposed on the needle hub NH just below the needle cover NC to lever the needle cover NC. Specifically, the extractor 10 is disposed in a gap G formed between a lip L of the needle cover NC and a base J of the needle hub NH.

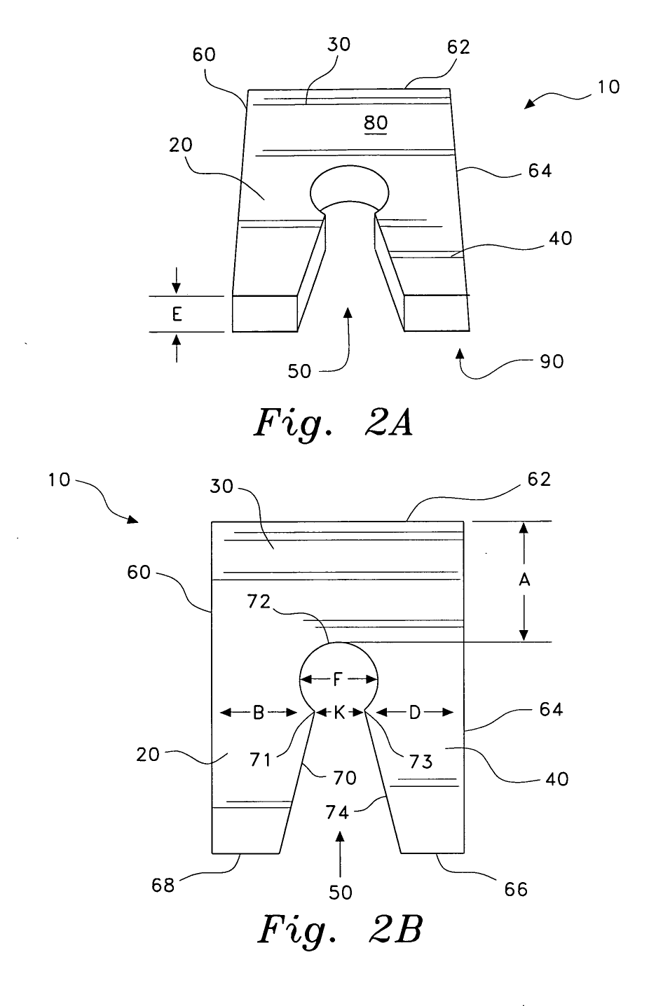

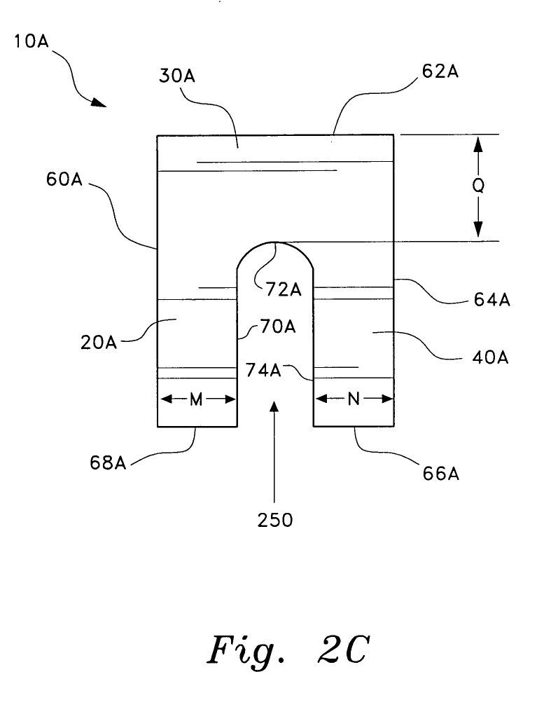

Viewing FIG. 2A in conjunction with FIG. 2B, the extractor 10 is shown in detail having a substantially planar body. In the present embodiment, the extractor 10 is square-like, however, the extractor 10 can take other shapes such as a circle or an oval and therefore is not be limited in shape. The extractor 10 has exterior sides 60, 62, 64 and 66, 68 and comprises a top surface 80, a bottom surface 90, two legs 20, 40, a connecting body 30 and a notched slot 50 disposed between the legs 20, 40.

As shown in FIG. 2A, surfaces 80, 90 are interchangeable and indistinguishable from each other and therefore permit a user to utilize the extractor 10 without regard to which surface 80 or surface 90 faces up on the syringe. The two legs 20, 40 each have a length defined by sides 60 and 64, respectively. The length of the legs 20, 40 should be longer than a diameter of the needle hub NH so the extractor 10 can surround and stay on the needle hub NH, see FIG. 1A.

Additionally, the extractor 10 has a height E, see FIG. 2A. The height E of the extractor 10 can be varied to fit any type of syringe depending on, and corresponding to, the size of the gap G between the lip L of the needle cover NC and the base J of the needle hub NH. Accordingly, extractor 10 can be thick, as shown in FIGS. 1A, 1B, 2A, 3A and 3B, or thin and plate-like.

Referring now to FIG. 2B, the notched slot 50 is defined by interior sidewalls 70, 74 and a C-shaped wall 72. The C-shaped wall 72 is disposed generally in the center of the extractor 10 having endpoints 71, 73. The C-shaped wall 72 has a general diameter F and a distance K separates the endpoints 71, 73. The distance between the sidewalls 70, 74 varies as it tapers from sides 68, 66, respectively, to endpoints 71, 73, respectively.

Sidewalls 70, 74 are situated to lie in an oblique orientation from endpoints 71, 73 to sides 68, 66, respectively and define a generally V-shaped path. Consequently, the generally V-shaped path tapers into the C-shaped wall 72 thereby defining the shape of the notched slot 50. The diameter F of the C-shaped wall 72, the distance K between endpoints 71, 73 and distance between the sidewalls 70, 74 can be made to vary according to the type of syringe being used and the curvature of the needle hub NH.

To insert the syringe through the notched slot 50 of the extractor 10, the needle hub NH on the syringe must slide through the generally V-shaped path against sides 70, 74, squeeze past endpoints 71, 73 and rest within the C-shaped wall 72. In order for the needle hub NH to squeeze past endpoints 71, 73, distance K must not be so small that it will not yield to pressure applied by the needle hub NH to endpoints 71, 73. Once the needle hub NH is within the C-shaped wall 72, endpoints 71, 73 surround the needle hub NH to permit the extractor 10 to remain on the syringe.

The legs 20, 40 are mirror images of each other and therefore have the same dimensions. Leg 20, at its widest part, has a width B defined between exterior side 60 and endpoint 71 of the C-shaped wall 72; leg 40, at its widest part, has a width D defined between exterior side 64 and endpoint 73 of the C-shaped wall 72. Widths B and D should have the same dimensions.

The connecting body 30 has a width defined by the exterior side 62 and a general length A defined between exterior side 62 and the C-shaped wall 72. In the preferred embodiment, length A of the connecting body 30 is greater than each the width B of leg 20 and width D of leg 40. Therefore the length A of the connecting body 30 serves as the region of the extractor 10 that is manipulated by the user.

Although extractor 10 is shown in the preferred embodiment having the notched slot 50, the slot 50 of the extractor 10 may take other shapes. For example, as shown in FIG. 2C, an extractor 10A can have a substantially U-shaped slot 250. Extractor 10A will have corresponding parts to the extractor 10, such as legs 20A, 40A, connecting body 30A, exterior sides 60A, 62A, 64A, 66A, 68A. Also, surfaces of the extractor 10A, like extractor 10, are indistinguishable from each other. The slot 250 has parallel interior sides 70A, 74A and a semicircular wall 72A. Legs 20A, 40A have a width M, N, respectively, and connecting body 30A has a length Q. Like extractor 10, widths M, N is smaller than length Q of the connecting body 30A.

The extractor 10, 10A is used to help ease the removal of the needle cover NC and reduce problems associated with removing the needle cover NC from the needle hub NH. One problem associated with removing the needle cover NC from the needle hub NH is damage incurred to the needle N when repeatedly trying and failing to remove the needle cover NC from the syringe. Another major problem is the increased likelihood of contaminating the needle N, the protuberance R and consequentially the medication that will be dispensed from the syringe.

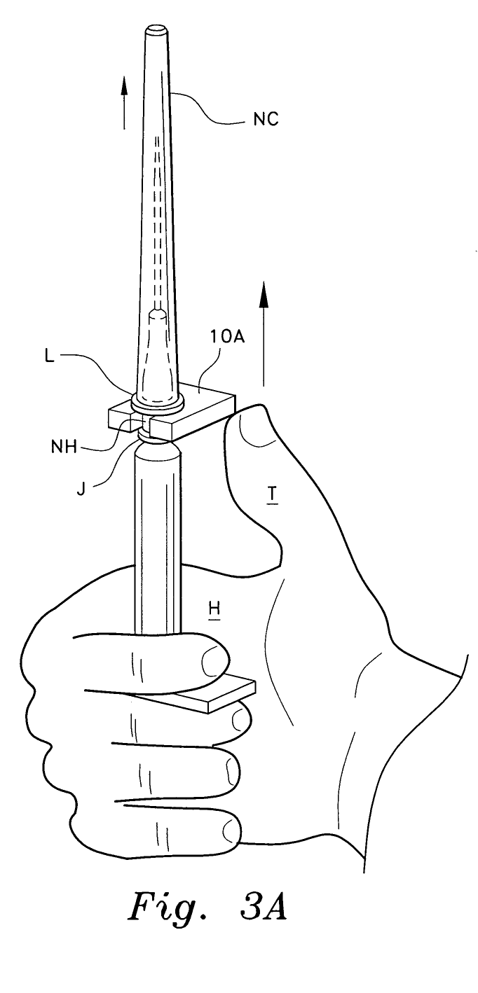

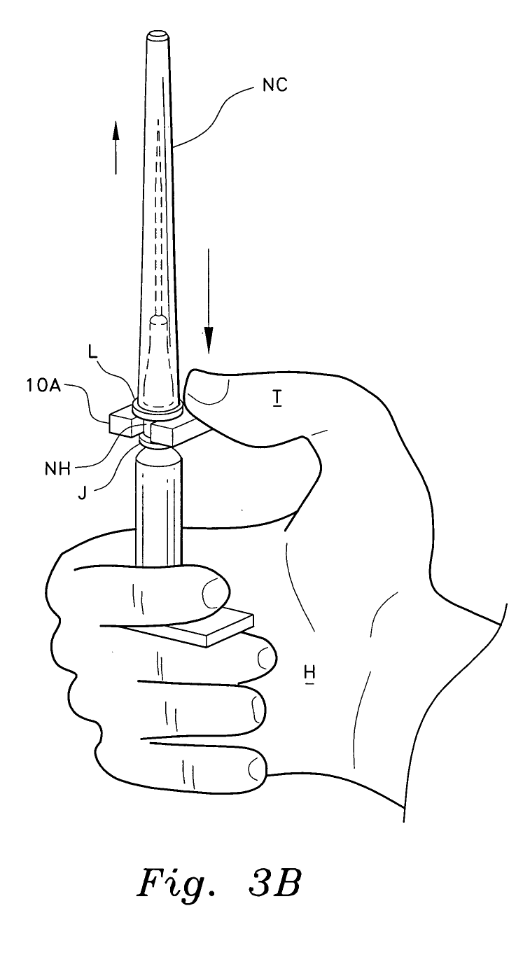

The needle cover NC is shown being removed from the syringe using extractor 10A, see FIG. 3A. Though described with extractor 10A, the needle cover NC may also be removed from the syringe in the same manner using extractor 10. With the extractor 10A disposed on needle hub NH, the user holds the syringe in one hand H using their thumb T to apply upward force on the width A of the connecting body 30A, to dislodge the needle cover NC up, off of the needle hub NH. Since the surface of the extractor 10A are interchangeable and indistinguishable, the user has the option of applying downward force on the width Q of the connecting body 30A to dislodge the needle cover NC from the syringe, see FIG. 3B.

Regardless of whether the user decides to apply upward or downward force on the extractor 10, 10A, only one hand is required to apply pressure on the extractor 10, 10A. Therefore, the user may utilize their free hand to hold the needle cover NC as it is being dislodged to prevent the needle cover NC from becoming a projectile object.

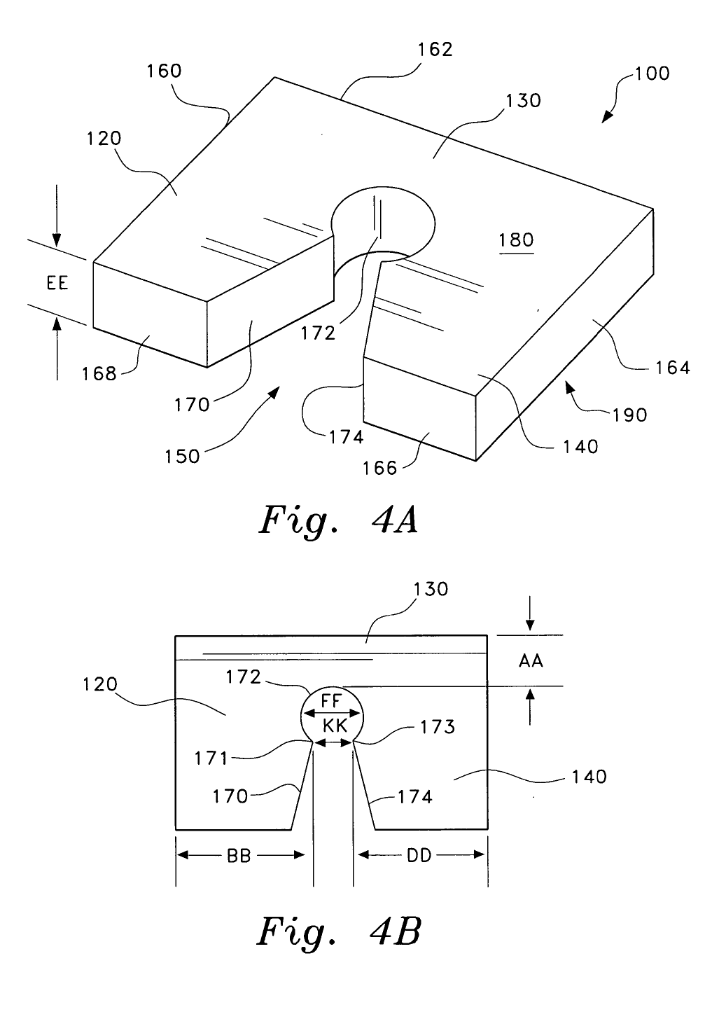

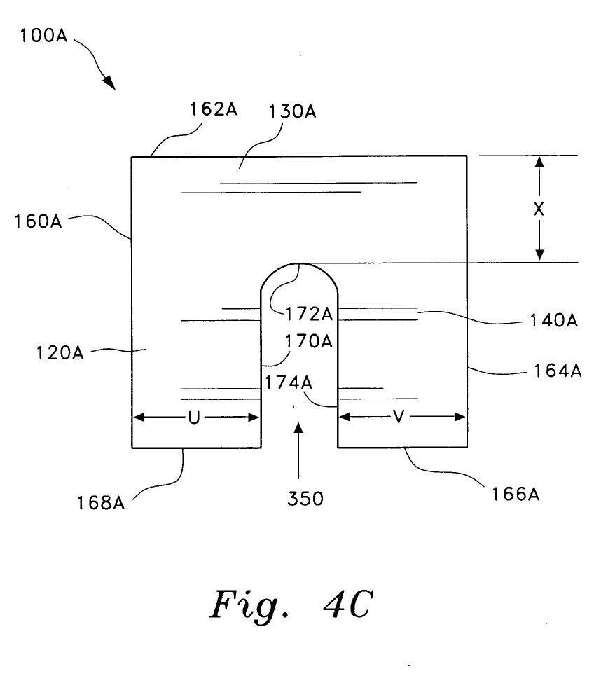

FIGS. 4A and 4B shows a needle cover extractor 100, which is an alternative embodiment to the needle cover extractor 10. The extractor 100 is generally rectangular in shape and has a substantially planar body. Again, as with the preferred embodiment extractor 10, extractor 100 may take other shapes such as a circle or an oval. The extractor 100 has a top surface 180, a bottom surface 190, exterior sides 160, 162, 164 and 166, 168, two legs 120, 140, a connecting body 130 and a notched slot 150 that defines legs 120 and 140 and the connecting body 130.

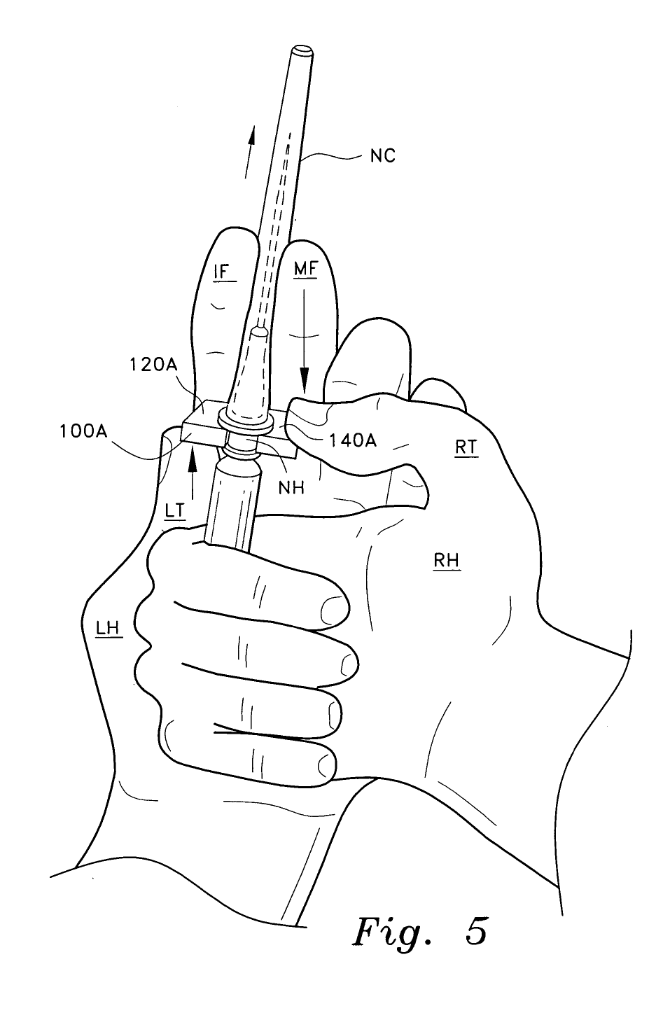

The extractor 100 is similar to the preferred embodiment extractor 10 in many ways. For example, extractor 100 has a height EE that can vary according to the gap G on the needle hub NH. The extractor 100 may be utilized on the syringe without regard to which surface 180 or 190 is facing up. Also, the legs 120, 140, each having a length defined by exterior side 160, 164, respectively, are longer than the diameter of the needle hub NH so that the extractor 100 can surround and stay on the needle hub NH, see FIG. 5.

Referring now to FIG. 4B, connecting body 130 has a length AA, leg 120 has a width BB and leg 140 has a width DD. Widths BB and DD are each greater than the length AA of the connecting body 130. Consequentially, the legs 120, 140 serve as the region where the user can manipulate the extractor 100. Still referring to FIG. 4B, the notched slot 150 is located in the center of the extractor 100 and is formed by interior sides 170, 174 and a C-shaped wall 172. The C-shaped wall 172 has endpoints 171, 173 from where sides 170, 174 meet and lie in an oblique orientation to sides 168, 166, respectively. The oblique orientation of sidewalls 170, 174 defines a generally V-shaped path to the C-shaped wall 172.

Due to the oblique orientation of sidewalls 170, 174, distances between the sidewalls 170, 174 vary with the distance at the exterior sides 168 and 166 being broader than the distance at the endpoints 171, 173. A distance KK separates endpoints 171 and 173 and a diameter FF defines the C-shaped wall 172. Diameter FF, distance KK and height EE, can all vary according to the particular type of syringe being used, the gap G and the diameter of the needle hub NH.

Extractor 100A shows an alternative arrangement the slot 50 of extractor 100, see FIG. 4C. Extractor 100A has a substantially U-shaped slot 350 with corresponding parts to the extractor 100, such as legs 120A, 140A, connecting body 130A, exterior sides 160A, 162A, 164A, 166A, 168A. Also, surfaces of the extractor 100A, like extractor 100, are indistinguishable from each other. The slot 350 has parallel sides 170A, 174A and a semicircular wall 172A. Legs 120A, 140A have a width U, V respectively, and connecting body 30A has a length X. Like extractor 100, length X of the connecting body 130A is smaller than width U of leg 120A and width V of leg 140A.

The user may simultaneously apply upward and downward force on the legs 120A, 140A in order to pry the needle cover NC loose from the needle hub NH, see FIG. 5. Removal of the needle cover NC is described in FIG. 5 using extractor 100A, however, the needle cover NC may also be removed from the syringe in the same manner using extractor 100. To remove the needle cover, NC, the user may cradle the syringe body SB in the right hand RH with the right thumb RT disposed above leg 140A to apply downward force on the extractor 100A. Simultaneously, the left hand LH will surround the right hand RH with the left thumb LT positioned below leg 120A to push the extractor 100A up. The needle cover NC can then be held between the first or index finger IF and the middle finger MF of the left hand to prevent the needle cover NC from becoming a projectile object.

The needle cover NC may be dislodged by reversing the grip so the left hand LH cradles the syringe and the right hand RH grasps the needle cover or the needle cover NC may be removed in any other number of ways. Alternatively, the user may decide to only apply force on one leg 120A or 140A of the extractor 100A to pry the needle cover NC from the needle hub NH.

Extractors 10, 10A, 100, 100A are preferably manufactured from a single piece of material. For example, plastics may be used to form the extractor 10, 100 by injection molding or using other techniques well known in the art. Plastic provides the extractors 10, 10A, 100, 100A with a range of consistencies. Preferably, the extractors 10, 10A, 100, 100A have a semi-rigid consistency. The extractors 10, 10A, 100, 100A, can alternatively be manufactured using other material as well, such as a sturdy rubber.

It is to be understood that the present invention is not limited to the embodiments described above, but encompasses any and all embodiments within the scope of the following claims.

1. A needle cover extractor dimensioned and configured to cooperate with a syringe and a needle cover, comprising:
a clip having a top surface; a bottom surface; a first leg having a width and a length; a second leg having a width and a length; and a connecting body having a width and a length, where the connecting body is disposed between the first leg and the second leg to form a slot dimensioned and configured for engaging a needle hub of a syringe.

2. The needle cover extractor according to claim 1, wherein said length of the connecting body is greater than the width of the first leg and the width of the second leg.

3. The needle cover extractor according to claim 1, wherein said width of the first leg and the width of the second leg are each greater than the length of the connecting body.

4. The needle cover extractor according to claim 1, wherein the legs each have a length that is longer than the diameter of the needle hub.

5. The needle cover extractor according to claim 1, wherein said clip has a substantially planar body.

6. The needle cover extractor according to claim 1, wherein said clip is made of a semi-rigid material.

7. The needle cover extractor according to claim 1, wherein said clip is made of a single piece of material.

8. The needle cover extractor according to claim 1, wherein said clip is made of plastic.

9. The needle cover extractor according to claim 1, wherein said top surface and said bottom surface are indistinguishable from each other, and interchangeable, permitting the clip to be placed on the needle hub without regard to a predetermined orientation.

10. The needle cover extractor according to claim 1, wherein said slot is notched comprising a V-shaped path that tapers into a C-shaped opening.

11. The needle cover extractor according to claim 1, wherein said slot is U-shaped.

12. The combination of a syringe, needle and a needle cover extractor, comprising:
a syringe having a needle connected to the syringe at a needle hub and a needle cover disposed over the needle and lodged on the needle hub; and
a clip having a substantially planar body, having a top surface; a bottom surface; a first leg having a width and a length; a second leg having a width and a length; and a connecting body having a width and a length, where the connecting body is disposed between the first leg and the second leg to form a slot dimensioned and configured for abutting the needle hub.

13. The combination according to claim 12, wherein said length of the extractor connecting body is greater than the width of the first leg and the width of the second leg.

14. The combination according to claim 12, wherein said width of the extractor first leg and the width of the extractor second leg are each greater than the length of the extractor connecting body.

15. The combination according to claim 12, wherein the extractor legs each have a length that is longer than the diameter of the needle hub.

16. The combination according to claim 12, wherein said extractor clip is made of a single piece of material.

17. The combination according to claim 12, wherein said extractor clip is made of plastic.

18. The combination according to claim 12, wherein said extractor top surface and said extractor bottom surface are indistinguishable from each other, and interchangeable, permitting the clip to be placed on the needle hub without regard to a predetermined orientation.

19. The needle cover extractor according to claim 12, wherein said slot is notched comprising a V-shaped path that tapers into a C-shaped opening.

20. The needle cover extractor according to claim 12, wherein said slot is U-shaped.
